(19) United States
(12) Patent Application Publication
(10) Pub. No.: US 2006/0099669 A1
(43) Pub. Date: May 11, 2006

(54) High throughput assay system

(76) Inventor: Larry Hryshko, Manitoba (CA)

Correspondence Address: Ade & Company, 1700-360 Main Street, Winnepeg, MB, R3C 3Z3, CA

(21) Appl. No.: 10/530,868

(22) PCT Filed: Oct. 9, 2003
(86) PCT No.: PCT/CA03/01516

§ 371 Date: Nov. 16, 2005

Related U.S. Application Data
(60) Provisional application No. 60/416,912, filed on Oct. 9, 2002.

(51) Int. Cl. C12M 1/34; C12Q 1/02

(52) U.S. Cl. 435/29; 435/287.1; 435/293.1

(57) ABSTRACT

A device and a method that permits rapid application of experimental solutions to both (i.e. intracellular and extra-cellular) surfaces of a membrane patch is described. In one embodiment, this is accomplished by mounting a membrane patch on a hole through the side of a hollow tube such that one surface can be readily perfused on the outside of the tube while simultaneously perfusing the inside of the tube. Thus, by measuring changes in membrane traffic using any of a variety of means known in the art, it is possible to determine the effect of test compounds presented to the intracellular and/or extracellular surface on membrane traffic. As can be seen, the instant device and method have the advantage of allowing both sides of the membrane to be accessed simultaneously, as described below. This is in contrast to existing patch clamp techniques where only a single membrane surface is readily accessible

### FIELD OF THE INVENTION

The present invention relates generally to the field of biochemical assays. More specifically, the present invention relates to a method of electrophysiological characterization of membrane-compound interactions.

### BACKGROUND OF THE INVENTION

The development of the patch clamp technique and its application towards investigating fundamental properties of ion channels is credited to Erwin Neher and Bert Sakmann, who were jointly awarded the Nobel Prize in Physiology or Medicine in 1991. This powerful approach constitutes one of the principal means of investigating ion channels, carriers, and transporters in biological membranes in use today. Furthermore, this technique is widely employed to investigate the actions of pharmacological agents, or discover new chemical entities, that target these classes of molecules.

Numerous pharmacological agents in current use target specific molecules known as ion channels, ion carriers, or transporters. Often, the agent will affect the functionality or activity of a specific type of these molecules. Cardiac antiarrhythmic drugs and several neurologically active agents typify these types of agents.

The patch clamp technique is an extremely versatile approach to investigate the actions of specific molecules. It can provide rich kinetic details of the functional activity even at the level of a single transport molecule. It is also widely employed to characterize novel pharmacological agents with respect to identifying efficacy, potency, mechanism of action, etc. A major limitation, however, is that patch clamp techniques remain labor intensive. Furthermore, for the purpose of high through-put screening, existing patch clamp techniques only provide rapid access to a single membrane surface.

In Cell Attached Patch Clamp Recordings, a patch electrode is sealed onto a cell membrane and electrophysiological recordings are made from the ion channel(s) within the patched region. Typically, the solutions within the pipette are chosen to isolate a particular class of ion channel or transporter and the cellular constituents remain “largely” intact or are systematically altered to accomplish a desired feature. For example, depolarizing the cell with KCI is commonly employed in efforts to eliminate or reduce the transmembrane potential. Also, pipettes may be internally perfused providing some capability for changing the extracellular environment. A common limitation is that solutions are changed very slowly (e.g. minutes) and incompletely as the original pipette solution mixes with the added perfusate.

In Excised Patch Clamp Recording: Inside-Out and Outside-Out Configurations, a pipette is sealed onto a membrane surface. Once a seal (typically gigohm resistance) is obtained, the membrane patch is excised from the cell, commonly by mechanically retracting the pipette from the cell. Two possible configurations are employed. Most commonly, inside-out patches are employed where the extracellular surface of the patch is exposed to the pipette solution and the intracellular surface is readily accessible to perfusion. Less commonly, outside-out patches are employed where the orientation is opposite to the above. The advantage of this latter configuration is that access to the extracellular surface is readily available. In both variations of this technique, it is possible to combine these patch clamp configurations with pipette perfusion, albeit with the inherent limitations of slow and incomplete changes in solution composition. Thus, only by combining both variations of this method can rapid access to both membrane surfaces be achieved, and rapid access to both membrane surfaces of a single membrane patch is not possible.

Whole Cell Attached Patch Clamping involves a pipette sealed onto a membrane surface and a patch that is subsequently disrupted providing electrical continuity with the patch pipette. Here, the entire cell membrane can be voltage clamped, the pipette contents slowly diffuse into the cell, and solutions can be rapidly applied to the extracellular surface. This technique is commonly used to screen compounds that exert their effects extracellularly or those that can cross cell membranes. Once again, it is of note that this method does not provide rapid access to the intracellular surface.

Clearly, a high-throughput screening method which allows analysis of the interaction between targets and compounds of interest on both sides of a membrane simultaneously is needed. Moreover, the fundamental characterization of these transport molecules would be greatly facilitated by having the capability of accessing both membrane surfaces simultaneously.

### SUMMARY OF THE INVENTION

According to a first aspect of the invention, there is provided a high-throughput assay device comprising:

a hollow tube having sidewalls defining an inner cavity, said inner cavity for passing a first flowable fluid therethrough;

open ends; and

an opening extending through a sidewall, said opening for mounting a membrane thereon.

According to a second aspect of the invention, there is provided a method of identifying a compound that alters membrane traffic comprising:

providing a high-throughput assay device comprising:
a hollow tube having sidewalls defining an inner cavity, said inner cavity for passing a first flowable fluid therethrough;
open ends; and
an opening extending through a sidewall, said opening for mounting a membrane thereon;

mounting a membrane patch onto the opening;

flowing a first flowable fluid containing a test compound through the inner cavity;

flowing a second flowable fluid over an outer surface of the device; and

determining whether the test compound increases or decreases traffic (eg. ionic currents, transported molecules) across the membrane patch.

According to a third aspect of the invention, there is provided a method of identifying a compound that alters membrane traffic comprising:

providing a high-throughput assay device comprising:
a hollow tube having sidewalls defining an inner cavity, said inner cavity for passing a first flowable fluid therethrough;
open ends; and
an opening extending through a sidewall, said opening for mounting a membrane thereon;

mounting a membrane patch onto the opening;

flowing a first flowable fluid through the inner cavity;

flowing a second flowable fluid containing a test compound over an outer surface of the device; and

determining whether the test compound increases or decreases traffic across the membrane patch.

According to a fourth aspect of the invention, there is provided a membrane traffic modulator isolated according to either of the above-described methods.

### BRIEF DESCRIPTION OF THE DRAWINGS

FIG. 1 shows the device in perspective and is an example of excised patch perfusion. The bold arrows show the direction of perfusion.

FIG. 2 shows the device with a cell mounted thereon and is an example of cell attached perfusion. The bold arrow shows the direction of perfusion.

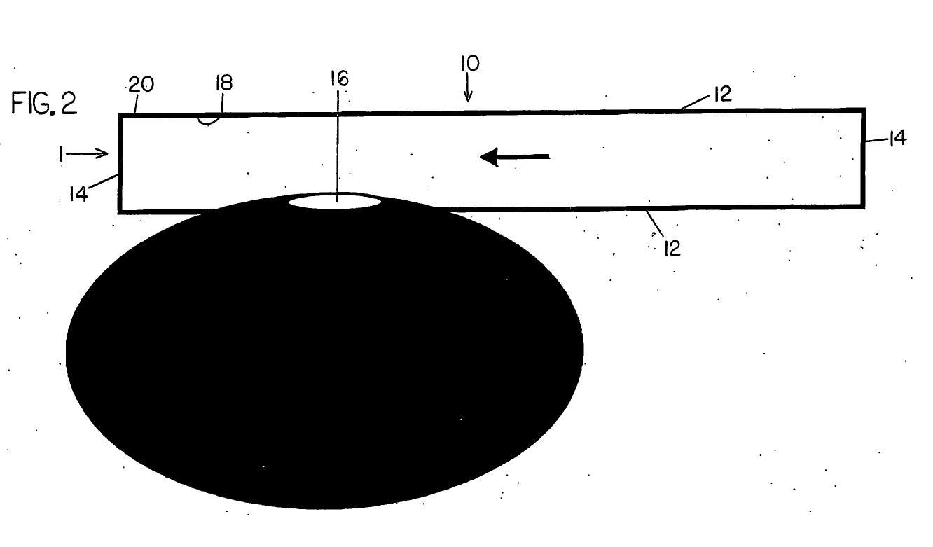

FIG. 3 shows the device in use.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

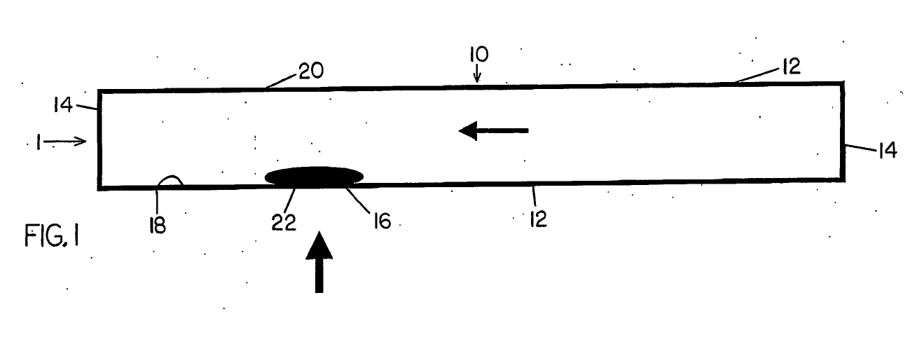

Described herein is a device and a method that permits rapid application of experimental solutions to both (i.e. intracellular and extracellular) surfaces of a membrane patch. In one embodiment, this is accomplished by mounting a membrane patch on a hole through the side of a hollow tube such that one surface can be readily perfused on the outside of the tube while simultaneously perfusing the inside of the tube. As will be appreciated by one of skill in the art, in this arrangement, the perfusate in the inside of the tube is isolated from the perfusate outside the tube by the walls of the tube and the membrane patch. As a consequence, compounds, for example, molecules, ions and peptides, can only cross from one perfusate to the other through the membrane. Thus, by measuring changes in membrane traffic using any of a variety of means known in the art, it is possible to determine the effect of test compounds presented to the intracellular and/or extracellular surface on membrane traffic. As can be seen, the instant device and method have the advantage of allowing both sides of the membrane to be accessed simultaneously, as described below. Schematically, this arrangement is shown in FIG. 1. This is in contrast to existing patch clamp techniques where only a single membrane surface is readily (and rapidly) accessible.

Thus, referring to the drawings, a high-throughput assay device 1 comprises a hollow tube 10. The tube 10 has sidewalls 12, two open ends 14, an interior surface 18 and an exterior surface 20. The sidewalls 12 includes at least one opening 16 arranged for mounting a membrane thereon, as described below. Thus, the sidewalls define an inner cavity for passing a flowable fluid or perfusate therethrough, as discussed below.

It is of note that as used herein, “tube” does not refer solely to cylindrical structures, but refers to a variety of geometries which may be used within the instant invention.

In use, a membrane patch 22 is mounted onto the high-throughput assay device 1 such that the membrane patch forms a gigaohm seal with the sidewalls 12 surrounding the opening 16. The open ends 14 are then connected to hoses or tubing or otherwise arranged such that the interior 18 of the tube 10 is isolated from the exterior 20 of the tube. A first flowable fluid or perfusate is passed through the interior 18 of the tube and a second flowable fluid or perfusate is passed across the exterior 20 of the tube 10.

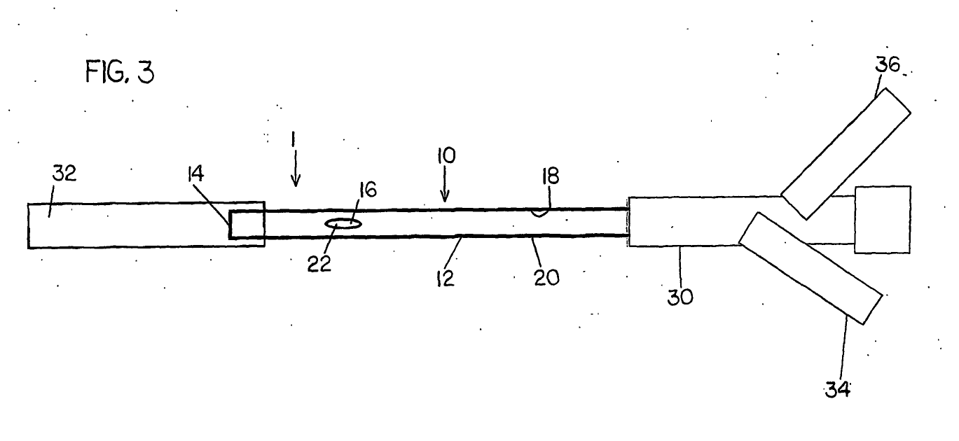

Specifically, referring to FIG. 1, the membrane patch 22 is mounted onto the high-throughput assay device 1 as described above. In some embodiments, shown in FIG. 3, the high-throughput assay device 1 with the membrane patch mounted thereon is connected to a pipette holder 30 and tubing 32. The pipette holder 30 includes a suction port 34 for withdrawing fluids and a perfusion port 36 for administering or delivering the first flowable fluid to the high-throughput assay device.

As discussed below, the first flowable fluid and/or the second flowable fluid contain at least one test compound. The test compound is then presented to either the inner surface or the outer surface of the membrane as the fluid flows, either through the tube or along the outside of the tube. The effect that the test compound has on membrane traffic, that is, whether ionic or substrate concentrations and/or ion traffic across the membrane increases or decreases or if other molecules including in some instances the test compound itself cross the membrane can be determined using means well known in the art. In this manner, test compounds can be assayed for example for their effect on ion channels, ion carriers and transporters on either side of a membrane of interest. Moreover, the fundamental transport characteristics of any ion channel, ion carrier, or ion transporter would be greatly facilitated by the application of this technique. As discussed herein, any suitable membrane may be used in the instant invention.

Thus, in embodiments shown exemplarily in FIG. 3, the first flowable fluid is applied via the perfusion port 36 while the second flowable fluid is applied to the exterior 20 of the high-throughput assay device 1 wherein at least one of the fluids contains a test compound of interest as described herein. The first flowable fluid passes the interior of the hollow tube 10 to the tubing 32 which is arranged for suction and perfusion efflux. Thus, as described herein, the first flowable fluid contacts or is exposed to one side (interior relative to the device 1) of the membrane patch 22 while the second flowable fluid contacts or is exposed to the other (exterior relative to the device 1) side of the membrane patch 22. As will be appreciated by one of skill in the art, once the assay has been completed, either the second flowable fluid and/or the first flowable fluid can be collected (for example, from the tubing 32) and assayed or analyzed for changes therein which correspond to or are indicative of a change in membrane traffic using any of a variety of means well known in the art.

The instant invention overcomes limitations in conventional patch clamping that hinder electrophysiological characterization of membranes. Specifically, using conventional techniques, it is only possible to rapidly access a single membrane surface for any given patch clamp configuration. Furthermore, to address questions concerning membrane sidedness of a particular effect, two membrane patches were required, again with the intrinsic limitations of slow alteration of one membrane surface. The instant invention alleviates these limitations by providing rapid access to both membrane surfaces, as discussed below. In one embodiment, capillary tubes were utilized in which a small (10-150 micrometer) hole was micro-machined into the side of the glass tube using lasers.

For electrophysiological purposes, pipettes are generally constructed of different glass types or quartz. Glasses are generally selected based on their thermal and electrical properties as these impact the shapes of pipette tips that can be produced and the electrical noise characteristics of the recordings. In general, this technique is directly applicable to any glass type, including quartz.

Other types of supports could be utilized, including polymers such as plastics and the like, including those polymers not routinely used for electrophysiology. The advantages of these supports include ease of hole preparation and a greater variety of dielectric properties.

The above-described device can be used in combination with electrophysiological devices known in the art to measure the effect of analytes on membrane traffic across the membrane patch. Specifically, analytes of interest may be placed in either the first flowable fluid, the second flowable fluid or both. By monitoring changes in membrane traffic, for example, an increase or decrease in ionic movements across the membrane patch or crossing of the membrane by an analyte, the effect of the analyte can be ascertained. In this manner, a variety of analytes, for example, chemical compounds, peptides and the like, can be screened for having an effect on membrane traffic. Furthermore, both sides of the membrane patch can be screened simultaneously. Thus, the instant device can be used to screen analytes for effects on, for example, ion channels, ion carriers and ion transporters.

The equipment employed for these experiments included an Axon Instruments™ 200A patch clamp amplifier, Axon Instruments pCLAMP software and a Narishige™ micromanipulator. Oocytes expressing the canine cardiac NCX1.1 exchanger were employed. Following removal of the vitelline layer by mechanical dissection, oocytes were placed in a Delrin recording chamber. A Drummond Scientific™ N-51A borosilicate glass pipette, which had an approximately 30 micrometer hole laser-machined into its side 1 cm from the open end, was connected to the headstage of the patch clamp amplifier. A silicon tube was connected to the open end of the pipette to permit mechanical constriction during the application of suction. The pipette holder was custom-fabricated to permit pipette perfusion from a variety of solutions and the selected perfusate flowed directly out the end of the silicone tubing and was thus electrically isolated from the bath: The pipette was placed over the oocyte and suction was applied from the suction port of the pipette holder while constricting the silicon tubing at the open end of the pipette. Once a gigaohm seal was obtained, the membrane patch was excised by retraction of the pipette from the oocyte. The pipette was then placed in a grooved platform, after which perfusion of both surfaces of the membrane patch could be accomplished using solenoid-controlled selection of perfusates.

In other embodiments, the oocyte is placed inside the pipette. Translocation of the oocyte to position it over the laser-machined hole was accomplished by applying positive pressure to either end of the pipette (i.e. the suction port or the silicon tubing at the end of the pipette). The hole in the pipette was then positioned over the end of another tube to permit suction for gigaohm seal formation. Once a seal was achieved, positive pressure was applied to the suction port of the pipette holder to propel the oocyte away from the hole. The membrane patch remained in the hole, again permitting simultaneous access of both membrane surfaces to perfusing solutions.

In both of the above examples, the clear advantage is that both membrane surfaces are accessible to perfusing solutions. Thus, one can monitor the activity of ion channels, ion carriers and ion pumps while applying chemical entities that target these specific molecules. This is particularly beneficial in situations where the sidedness of action of a compound is unknown. As the experimental variation employed to establish membrane orientation is very well defined (i.e. by obtaining the membrane seal while placing the cell either inside or outside the pipette), the sidedness of action of particular agents is readily and conveniently established. Furthermore, the technique is directly amenable to high-throughput screening of compounds as only small volumes of solution are required to perfuse either surface and a large number of compounds can be applied sequentially. In our particular apparatus, we have the capability of applying 20 unique solutions to the bath solution and 8 unique solutions to the pipette. Obviously, the number of experimental solutions that could be applied to either surface could be easily increased and the technique is directly amenable to automation.

In the above examples, a borosilicate glass tubing was utilized. As will be appreciated by one of skill in the art, any type of capillary tubing could be employed for this electrophysiological purpose, provided that electrical isolation occurs between the bath and pipette solutions. For example, any types of glass or non-metallic, non-electrically-conductive tubing would be suitable. Furthermore, a variety of methods could be utilized to obtain the hole in the side of the tubing. In the examples described above, this was accomplished using laser machining. However, this could also be accomplished using drilling procedures, chemically (for example, by dissolving a hole in glass with HF acid), molding procedures, as well as other suitable means known in the art. In embodiments wherein the techniques are applied for non-electrophysiological purposes (e.g. fluorescence measurements) there is no need to employ non-electrically conductive tubing to obtain the membrane patch. Furthermore, there is no obvious limitations on the size of holes to be employed, which would be appropriately selected based on the particular experimental needs. Finally, the technique can also be applied to whole cells, rather than isolated membrane patches.

The screening of pharmaceutical compounds is one application for this technique. In many cases, pharmaceutical compounds, for example, toxins, chemicals, peptides and the like, will only exert their effects when exposed to a particular membrane surface. Other compounds can access their target from either surface, a capability typical for lipophilic agents. With respect to screening compounds using electrophysiological techniques, however, one typically has to anticipate a sidedness as existing techniques only permit ready access to a single membrane surface. This invention alleviates this restriction by permitting rapid access to both membrane surfaces, a benefit that should accelerate the ability to screen substances without prior knowledge of their site of action. In this regard, the instant invention is superior to existing means of characterizing agents where the specific site of action is unknown.

For example, the technique is particularly well suited to the investigation of membrane channels, carriers, or channels that are heterologously expressed in Xenopus laevis oocytes. This provides the opportunity to examine a specific target of choice (expressed in the oocyte). A patch of membrane is then excised, the activity of the target of interest is monitored, and agents can be applied to either or both membrane surfaces to examine for a specific effect. However, the technique is also amenable to rapidly screening heterologously expressed or native channels in other membrane environments. In other words, membrane preparations from any convenient cell line can be employed where ion channels, ion carriers, and/or ion transporters can be heterologously expressed. In addition, in the absence of patch excision, it is possible to obtain the cell attached configuration, and rapidly screen agents by applying them to the extracellular surface of the patch. Alternatively, native transporters can be examined either as excised or cell attached patches, In all cases, the primary advantage is the rapid accessibility of either or both membrane surfaces depending upon experimental requirements.

As will be appreciated by one of skill in the art, this method can be used to examine ion channels (for example, L-type calcium channels), ion carriers (e.g. sodium-calcium exchanger), and ion pumps (sodium, potassium ATPase).

As will be appreciated by one of skill in the art, any suitable membrane may be used in the instant invention, provided that there is compatibility with cell size and hole size.

As will be apparent to one of skill in the art, electrophysiological experiments involve the alteration or perturbation of solution composition on a particular membrane surface to gain insight into some characteristic of an ion channel's behavior. This invention essentially doubles the experimental capabilities as both membrane surfaces are now readily (and rapidly) accessible, whereas this capability was not previously available with any existing techniques. That is, it is known in the art that it is possible to switch solutions at both membrane surfaces, albeit very slowly (i.e. minutes) and incompletely (i.e. by adding something to the inside of a cell or pipette, although the effect on concentration is unclear, as it is impossible to tell if you are simply diluting or concentrating something into an unknown volume). However, in the instant invention, changes are very rapid (fraction of seconds to seconds) and complete as the solutions are changed in their entirety.

One benefit of the above invention is the ability to access both the intracellular and extracellular surface of a cellular membrane while recording electrophysiological data. Other suitable applications would include the simultaneous or independent acquisition of fluorescence, colorimetric, spectroscopic, or biochemical data while retaining simultaneous access to both membrane surfaces. The obvious advantage of this invention is that one can alter the intracellular and extracellular environments either independently or simultaneously depending upon the experimental intent.

As discussed above, the instant invention may be used in a high-throughput screen for identifying compounds which interact with targets of interest. Specifically, a screen for compounds which affect a specific target on either the inner or outer surface of a membrane of interest may be carried out. Accordingly, the invention also includes compounds identified using this method.

In the above-described examples, capillary tubes utilized had a 0.084 inch outer diameter by 0.064 inch inner diameter by 3 inches long. Through holes of various diameters (approximately 15 micrometers to 150 micrometers) were drilled through the wall of the glass approximately 1 cm from the tip using lasers. As will be appreciated by one of skill in the art however, these exact details are not critical for utilization of the technique and glass selection and through hole particulars should be selected based on experimental intent and convenience.

While the preferred embodiments of the invention have been described above, it will be recognized and understood that various modifications may be made therein, and the appended claims are intended to cover all such modifications which may fall within the spirit and scope of the invention.

1. A high-throughput assay device comprising:
a. hollow tube having sidewalls defining an inner cavity, said inner cavity for passing a first flowable fluid therethrough;
open ends; and
an opening extending through a sidewall, said opening for mounting a membrane thereon.

2. A method of identifying a compound that alters membrane traffic comprising:
providing a high-throughput assay device comprising:
a hollow tube having sidewalls defining an inner cavity, said inner cavity for passing a first flowable fluid therethrough;
open ends; and
an opening extending through a sidewall, said opening for mounting a membrane thereon;
mounting a membrane patch onto the opening;
flowing a first flowable fluid containing a test compound through the inner cavity;
flowing a second flowable fluid over an outer surface of the device; and
determining whether the test compound increases or decreases traffic across the membrane patch.

3. A membrane traffic modulator isolated according to the method of claim 2.

4. A method of identifying a compound that alters membrane traffic comprising:
providing a high-throughput assay device comprising:
a hollow tube having sidewalls defining an inner cavity, said inner cavity for passing a first flowable fluid therethrough;
open ends; and
an opening extending through a sidewall, said opening for mounting a membrane thereon;
mounting a membrane patch onto the opening;
flowing a first flowable fluid through the inner cavity;
flowing a second flowable fluid containing a test compound over an outer surface of the device; and
determining whether the test compound increases or decreases traffic across the membrane patch.

5. A membrane traffic modulator isolated according to the method of claim 4.
